(19) United States
(12) Patent Application Publication
(10) Pub. No.: US 2025/0257191 A1
(43) Pub. Date: Aug. 14, 2025

(54) DISPERSANT ADDITIVES AND METHODS OF MAKING AND USE THEREOF

(71) Applicant: Ecolab USA Inc., St. Paul, MN (US)

(72) Inventors: Kameswara R. Vyakaranam, Sugar Land, TX (US); Meng Kong, Shanghai (CN); Ashish Dhawan, Aurora, IL (US)

(21) Appl. No.: 18/289,061

(22) PCT Filed: Nov. 3, 2022
(86) PCT No.: PCT/CN2022/129410

§ 371 Date: Oct. 31, 2023

(51) Int. Cl. C08K 5/3445

(52) U.S. Cl. CPC C08K 5/3445

(57) ABSTRACT

Imidazoline additives, and methods of making and using the additives are described. The additives may be provided as concentrates for addition to process compositions comprising acrylic acid, methacrylic acid, salts of acrylic acid, salts of methacrylic acid, or other free radically polymerizable monomers to prevent fouling. The additives are made by reacting glycerol-restricted vegetable oil fatty acid with amine, wherein the glycerol-restricted vegetable oil fatty acid is at least partially purified to remove glycerol. The imidazoline additives may be further reacted to provide ammonium salts that may be added to aqueous compositions, for example as aqueous dispersants.

### TECHNICAL FIELD

The invention is directed to imidazoline additives, imidazoline ammonium salt additives, and methods of making and using the additives. The additives are derived from amine compounds and one or more of glycerol-restricted vegetable oil fatty acids, such as glycerol-restricted soybean oil fatty acid. The additives may be used as dispersants, for example in the synthesis or polymerization of unsaturated monomers, but may also find use as anticorrodents, lubricants, antifoaming agents, foaming agents, and release agents.

### BACKGROUND

A common method of production of acrylic acid and methacrylic acid is through gas phase catalytic oxidation of alkanes, alkenes, alkanols, or alkenals containing three to four carbon atoms. The reaction products from the oxidation process are separated, and (meth)acrylic acid is purified in the recovery and purification section. The design of the recovery and purification section varies in process equipment and operating conditions, but fundamentally it comprises extraction and distillation separations. In a generalized (meth)acrylic acid recovery and purification process, the effluent from the oxidation process is cooled in an absorber to remove the light components in the product effluent. Then, in the extraction column, (meth)acrylic acid is concentrated with a selected solvent to remove water, acetic acid, or both water and acetic acid. The crude (meth)acrylic acid stream is then purified of remaining extraction solvent and reaction by-products in succeeding distillation towers. Design variations exist with solvent selection.

(Meth)acrylic acids and many other addition-polymerization monomers containing unsaturated functionality are reactive monomers, and they tend to polymerize with any trivial change in environment. This is the case during the recovery and purification operations in manufacturing (meth)acrylic acid, where elevated temperatures accelerate the polymerization of the (meth)acrylic acid. Under such circumstances, the undesired polymerization may become so severe that polymer deposition fouls process equipment. Eventually equipment shutdown and cleaning is required for removal of the polymeric foulant. Conventionally, polymerization inhibitors are used in the manufacturing processes of the monomers to inhibit this undesired polymerization. Typical polymerization inhibitors are phenolic compounds, amine compounds, quinones, nitroxyl compounds and certain inorganic complexes. Phenothiazine (PTZ), hydroquinone (HQ) and monomethyl hydroquinone ether (MEHQ) are examples of widely used inhibitors. These inhibitors are designed to interrupt the polymerization reactions and prevent the formation of the polymer. However, none of the available polymerization inhibitors are efficient enough to completely eliminate undesired polymer formation. Even in the presence of these inhibitors, polymer formation and subsequent fouling is still substantial, so that periodic cleaning is a part of routine (meth)acrylic acid processes.

In industrial practice, dispersants may also be used in addition to polymerization inhibitors to improve fouling prevention. Dispersants often comprise molecules with an affinity for the foulant particle surface and good solubility in the liquid process stream. Unlike inhibitors, dispersants generally do not interfere with the polymerization reactions involved in foulant formation, Dispersant molecules prevent formed polymer particles from agglomerating, thereby keeping them suspended in the process medium. The selection of an effective dispersant remains an experimental art, because the utility of such a dispersant is highly dependent on the detailed nature of the foulant material and liquid medium, both of which are unique to a given process.

Batches of manufactured additive formulations are typically stored for some period of time before use or further processing. It is desirable that during storage and/or transport additive formulations are stable, for example to undue phase separation into separate liquids, to precipitation of solids and/or gels, to settling, or to other undesired phase separation. Additive formulations exhibiting such changes may require special treatment to return the additive formulation to its original state. It may not even be possible to return the additive formulation to its original state in a practical manner, and in its changed form the additive formulation may be unusable. For example, in additive formulations including precipitates, settled material may not be pumpable or pourable. In any case, undue phase separation of additive compositions during storage or transport is highly undesirable. In contrast, in some instances phase separation during actual use in an industrial process may be acceptable or even desired in cases in which the additive can still provide the desired function, e.g. dispersion in the case of a dispersant. In other instances, it may be desirable to protect against phase separation even during use in an industrial process.

For addition to industrial process streams or batches, additive formulations may be conveniently provided in a variety of forms such as in the form of a solution or dispersion in an organic solvent that is miscible in the process streams or batches. Since such solutions or dispersions of the additives are typically shipped and stored, it can be economical if the solutions or dispersions are relatively concentrated with respect to the additives, thereby avoiding the extra expense that shipping and storing a commodity such as organic solvent entails. Such solutions or dispersions provide flexibility in that they can easily be further diluted at a more suitable time such as at or close to point-of-use if so desired, while they can be used without further dilution if higher concentrations are desired.

Like the additives themselves, such solutions and/or dispersions or more diluted formulations of additives are desirably sufficiently stable during storage, shipping, and use, Solutions, dispersions, and/or more diluted formulations of additives are routinely stored and/or transported in unheated winter environments, where they attain temperatures of, for example, 0° C. or below, −10° C. or below, −20° C. or below, −30° C., or even −40° C. or below.

Accordingly, it would be an advantage if dispersant formulations and other additive formulations could be found that do not unduly phase separate and/or settle in storage, handling, or use, and that when provided in solutions and/or dispersions in organic solvents do not unduly separate, even when stored, handled, transported, or otherwise used at low temperatures.

### SUMMARY

We have discovered additive compositions with restricted glycerol content that are more stable to phase separation and to the formation of precipitates when provided as a composition comprising organic solvent, even at lower temperature such as minus 20° C. or lower. One or more additives derived from vegetable oil fatty acids such as soybean oil fatty acid are more stable to phase separation if at least a portion of the vegetable oil fatty acid content has a reduced glycerol content. In preferred mode of practice, additive compositions with improved stability incorporate one or more additives derived from glycerol-restricted soybean oil fatty acid. In other modes of practice, the formulations or one or more components thereof, may be processed to remove at least a portion of the glycerol content to thereby improve stability against phase separation.

We have also discovered that compositions of additives in organic solvent, when made from glycerol-restricted soybean oil fatty acid, are more stable to precipitation (i.e. exhibit less or no precipitation) when stored at temperatures such as 0° C. and −10° C. for three weeks than equivalent concentrates made from tall oil fatty acid.

In one aspect is provided an additive composition comprising a plurality of imidazoline compounds, a plurality of ammonium salts of the imidazoline compounds, or a combination thereof, wherein at least a portion of the imidazoline compounds comprises a reaction product derived from reactants comprising an amine and a glycerol-restricted soybean oil fatty acid, wherein the glycerol-restricted soybean oil fatty acid comprises a mixture of fatty acids and less than 0.1 percent by weight glycerol with respect to the weight of the glycerol-restricted soybean oil fatty acid, and wherein the amine has the formula NH₂—(CH₂)n-NH—R¹—OH, wherein R¹ is a divalent moiety comprising one or more C atoms and optionally one or more heteroatoms, and wherein n is 2 or 3.

For example, if the glycerol-restricted soybean oil fatty acid includes 0.06 parts by weight of glycerol and 100 parts by weight of soybean oil fatty acid, then the total weight of the glycerol-restricted soybean oil fatty acid is 100.06 parts by weight, and the glycerol content is 0.06/100.06×100%=0.06%. The plurality of imidazoline compounds and/or ammonium salts thereof function in some embodiments as a dispersant additive in the additive compositions, for example to help protect against fouling in processes incorporating unsaturated monomers.

In a further aspect is provided a concentrate comprising an additive composition, the additive composition comprising a plurality of imidazoline compounds, a plurality of ammonium salts of the imidazoline compounds, or a combination thereof, wherein at least a portion of the imidazoline compounds comprises a reaction product derived from reactants comprising an amine and a glycerol-restricted soybean oil fatty acid, wherein the glycerol-restricted soybean oil fatty acid comprises a mixture of fatty acids and less than 0.1 percent by weight glycerol with respect to the weight of the glycerol-restricted soybean oil fatty acid, and wherein the amine has the formula NH₂—(CH₂)n-NH—R¹—OH, wherein R¹ is a divalent moiety comprising one or more C atoms and optionally one or more heteroatoms, and wherein n is 2 or 3; and wherein the composition comprises about 0.1 parts by weight to 75 parts by weight of at least one organic solvent per 99.9 parts by weight to 25 parts by weight of the plurality of imidazoline compounds, the ammonium salts of the plurality of the imidazoline compounds, or the combination thereof. The plurality of imidazoline compounds, the ammonium salts of the imidazoline compounds, or the combination thereof may be dispersed or dissolved in the at least one organic solvent, the at least one organic solvent may be miscible with a process stream or batch, and the concentrate may accordingly disperse easily in the process stream or batch when added thereto, thereby imparting favorable properties to the process stream or batch such as reduced tendency to foul.

In a further aspect is provided a treated composition comprising an additive composition, the additive composition comprising a plurality of imidazoline compounds and/or a plurality of ammonium salts thereof, wherein at least a portion of the imidazoline compounds comprises a reaction product derived from reactants comprising an amine and glycerol-restricted soybean oil fatty acid, wherein the glycerol-restricted soybean oil fatty acid comprises a mixture of fatty acids and less than 0.1 percent by weight glycerol with respect to the weight of the glycerol-restricted soybean oil fatty acid, and wherein the amine satisfies the formula NH₂—(CH₂)n-NH—R¹—OH, wherein R¹ is a divalent moiety comprising one or more C atoms and optionally one or more heteroatoms, and wherein n is 2 or 3; and wherein the composition further comprises a free-radically polymerizable monomer. Such treated compositions may display a lower tendency to undesirably foul equipment that comes in contact therewith, for example when subjected to heat, than equivalent compositions lacking the imidazoline compounds and/or the ammonium salts thereof.

In a further aspect is provided a method of making an additive composition, the method comprising: (a) combining an amine and glycerol-restricted soybean oil fatty acid to provide a reaction mixture; and (b) heating the reaction mixture to form the additive composition, wherein the additive composition comprises a plurality of imidazoline compounds, wherein at least a portion of the imidazoline compounds comprises a reaction product derived from reactants comprising the amine and the glycerol-restricted soybean oil fatty acid, wherein the glycerol-restricted soybean oil fatty acid in step (a) comprises a mixture of fatty acids and comprises less than 0.1 percent by weight glycerol with respect to the weight of the glycerol-restricted soybean oil fatty acid in step (a), and wherein the amine in step (a) satisfies the formula NH₂—(CH₂)n-NH—R¹—Y, wherein R¹ is a divalent moiety comprising one or more C atoms and optionally one or more heteroatoms, wherein n is 2 or 3, and wherein Y is —NH₂ or —OH. The additive compositions may find use in a wide variety of applications, for example as antifoulants and/or dispersants for polymers.

In a further aspect is provided a use of an additive composition as one or more of a corrosion inhibitor, dispersant, lubricant, antifoam agent, antifoulant, and release agent. The additive composition comprises a plurality of imidazoline compounds, a plurality of ammonium salts of the imidazoline compounds, or a combination thereof, wherein at least a portion of the imidazoline compounds comprises a reaction product derived from reactants comprising an amine and a glycerol-restricted soybean oil fatty acid, wherein the glycerol-restricted soybean oil fatty acid comprises a mixture of fatty acids and less than 0.1 percent by weight glycerol with respect to the weight of the glycerol-restricted soybean oil fatty acid, and wherein the amine has the formula NH₂—(CH₂)n-NH—R¹—OH, wherein R¹ is a divalent moiety comprising one or more C atoms and optionally one or more heteroatoms, and wherein n is 2 or 3.

### DETAILED DESCRIPTION

Although the present disclosure provides references to various embodiments, persons skilled in the art will recognize that changes may be made in form and detail without departing from the spirit and scope of the application. Various embodiments will be described in detail with reference to the figures. Reference to various embodiments does not limit the scope of the claims attached hereto. Additionally, any examples set forth in this application are illustrative and are not intended to be limiting and merely set forth some of the many possible embodiments for the appended claims.

### Definitions

Unless otherwise defined, all technical and scientific terms used herein have the same meaning as commonly understood by one of ordinary skill in the art. In case of conflict, the present document, including definitions, will control. Methods and materials are described below, although methods and materials similar or equivalent to those described herein can be used in practice or testing of the present application. All publications, patent applications, patents and other references mentioned herein are incorporated by reference in their respective entireties and for all purposes.

As used herein, the terms “comprise(s),” “include(s),” “having,” “has,” “can,” “contain(s),” and variants thereof are intended to be open-ended transitional phrases, terms, or words that do not preclude the possibility of additional acts or structures. The singular forms “a,” “and” and “the” include plural references unless the context clearly dictates otherwise.

As used herein, the term “optional” or “optionally” means that the subsequently described material, condition, feature, event or circumstance may but need not occur, and that use thereof includes instances where the material, condition, feature, event or circumstance occurs and instances in which it does not.

As used herein, any recited ranges of values contemplate all values within the range and are to be construed as support for claims reciting any sub-ranges having endpoints which are real number values within the recited range. By way of example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5; and fractions thereof e.g. 1.5-3.5, 1.7-4.8, etc.

Herein, the combination of fatty acids that may be produced by hydrolysis of a single type of vegetable oil is referred to as “vegetable oil fatty acid”. Because a single type of vegetable oil comprises esters of a variety of fatty acids, hydrolysis of the vegetable oil produces a combination of fatty acids. For example, hydrolysis of soybean oil may produce “soybean oil fatty acid” in combination with byproduct glycerol. While used in the singular, soybean oil fatty acid contains a combination of different fatty acid compounds, not just a single fatty acid compound. Vegetable oils are natural products, and may vary in composition, for example with regard to the relative content of various fatty acid compounds in the form of esters. However, the types and ranges of each fatty acid compound in the vegetable oil fatty acid are generally characteristic of the type of vegetable oil from which the vegetable oil fatty acid may be derived. While “vegetable oil fatty acid” used in the singular refers to a combination of fatty acid compounds that may be produced by the hydrolysis of the namesake vegetable oil, “vegetable oil fatty acids” refers herein to a combination of fatty acids that may be produced by the hydrolysis of a combination of two or more types of vegetable oil.

As used herein, the term “glycerol-restricted” with respect to a material generally signifies that the material comprises no more than 0.1 percent by weight of glycerol with respect to the weight of that material (including glycerol if any). For example, “glycerol-restricted fatty amide” refers to a material comprising fatty amide(s), wherein the material comprises no more than 0.1 percent by weight of glycerol with respect to the weight of the glycerol-restricted fatty amide.

As used herein, the term “glycerol-restricted vegetable oil fatty acid” specifically refers to a mixture comprising no more than 0.1% by weight glycerol and no less than 99% by weight of fatty acids with respect to the weight of the mixture. Glycerol-restricted vegetable oil fatty acids may be made by various means. One commercially important means of making a glycerol-restricted vegetable oil fatty acid is by hydrolysis of the namesake vegetable oil and subsequent purification to remove glycerol, which is a byproduct of the hydrolysis. The types of fatty acid and their proportions in the glycerol-restricted vegetable oil fatty acid depend upon the composition of the vegetable oil hydrolyzed, especially the type of vegetable oil, that is the type of vegetable from which the vegetable oil was sourced. For example, while soybean oil as a natural product may vary in composition with respect to fatty acid esters, it may do so within ranges that are characteristic of soybean oil. However, in principle the glycerol-restricted vegetable oil fatty acid could also be produced by combining the correct types of fatty acid in the correct proportions and for which the cumulative glycerol content, if any, is sufficiently low such that the resultant mixture is glycerol-restricted as defined in accordance with the present invention. In such a mode of practice, some degree of purification to reduce the glycerol content would not be needed, since the fatty acid materials are suitably glycerol-restricted at the outset. Solely for the purposes of illustration, a glycerol-restricted soybean oil fatty acid could in a particular example comprise 10% by weight palmitic acid, 4% by weight of stearic acid, 23% by weight oleic acid, 51% by weight of linoleic acid, and 9% by weight of alpha-linolenic acid with respect to the total weight of fatty acids. However, such a glycerol-restricted soybean oil fatty acid could in principle be produced by combining 10 parts by weight palmitic acid, 4 parts by weight of stearic acid, 23 parts by weight of oleic acid, 51 parts by weight of linoleic acid, 9 parts by weight of alpha-linolenic acid, and any other components of the glycerol-restricted soybean oil fatty acid in which the glycerol is restricted as supplied or the mixture is purified to remove glycerol. Accordingly, glycerol-restricted vegetable oil fatty acid refers to a mixture that comprises a combination of fatty acids that is characteristic of that produced by the particular namesake vegetable oil.

As used herein, “hydrolyzed vegetable oil” and like terms including specific vegetable oil names refers to a composition that is produced by hydrolysis of the vegetable oil. For example, the composition comprising soybean oil fatty acid and glycerol that may be produced by the hydrolysis of soybean oil is termed herein “hydrolyzed soybean oil”.

As used herein, “(meth)acrylic acid” means methacrylic acid and/or acrylic acid.

As used herein, “preventing” includes inhibiting.

As used herein, the term “concentrate” generally refers to a composition in which the concentration of additive(s) is higher than the concentration of such additives after the composition is diluted for an intended use. A concentrate may be a solid, liquid, dispersion, gel, or gas. A concentrate may or may not include solvent until further diluted. In representative modes of practice, a concentrate may include 0 to 80 parts, or even 0 to 50 parts, or even 0 to 25 parts, or even 0 to 10 parts of solvent per 100 parts by weight of additive(s) prior to dilution with a solvent. If desired, a concentrate can be diluted by adding some amount of additional solvent, which may be the same and/or different than the solvent in the concentrate. By way of example, dilution may occur by adding 1 to 1000, or even 10 to 500, or even 10 to 100 parts by weight of additional solvent per 50 to 100 parts by weight of solvent in the concentrate. All reaction schemes herein are for the purpose of illustrating some reactants and some reaction products.

### Discussion

In commercial practice, imidazoline compounds derived from fatty acids have been used as dispersants or as other types of additives, for example in synthesis and processing of vinyl monomers such as (meth)acrylic acid and/or vinyl acetate. The imidazoline compounds may be made by reacting tall oil or tall oil fatty acid with amine compounds. Tall oil and tall oil fatty acid (TOFA) are convenient sources of fatty acids. Tall oil is a byproduct of the Kraft process of wood pulp manufacture. Typically crude tall oil contains rosins, which contain resin acids (mainly abietic acid and its isomers), a mixture of fatty acids (mainly palmitic acid, oleic acid and linoleic acid), fatty alcohols; sterols; and other alkyl hydrocarbon derivates. Tall oil fatty acid is a purified form of tall oil and contains a lower weight percentage of rosin (1-10 percent by weight) than tall oil. Tall oil fatty acid comprises fatty acids, predominantly oleic acid.

Tall oil fatty acid (TOFA) may be reacted with one or more amine compounds to produce TOFA imidazoline, a reaction product comprising a mixture of imidazoline compounds and other components resulting from resin acids in the TOFA. The reaction can be summarized as follows:

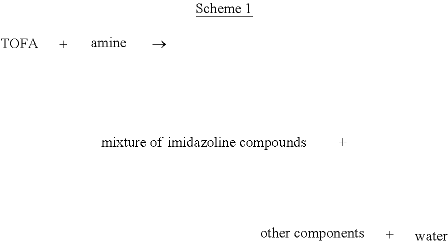

The reaction proceeds via intermediate amides, which may cyclize, for example, if the amine contains the group NH₂(CH₂)nNH—, wherein n is 2 or 3.

TOFA-derived imidazoline compounds may find use as dispersant additives, for example, in processing of vinyl monomers. Conveniently, additives may be provided as concentrates in an organic solvent. Such concentrates, either as is or in a diluted form, may be provided with high imidazoline concentrations, for example greater than 40 weight percent, 50 weight percent, 60 weight percent, 70 weight percent, 80 weight percent, or more than 90 weight percent of the imidazoline compounds with respect to the weight of the concentrate. Such concentrates may be stored, shipped, and provided to processing plants for addition to processing streams or batches therein. However, concentrates or even diluted forms of such TOFA-derived additives may be unstable when stored in low temperatures, for example 0° C. or less, −10° C. or less, or −20° C. or less, over a period of days or weeks, exhibiting precipitates, other phase separation, or other instability so that the affected composition cannot be easily redissolved or redispersed. For example dispersant compositions incorporating a reaction product comprising imidazoline compounds derived from TOFA and 2-(2-aminoethyl) ethanolamine (AEEA) exhibited precipitation after only a few days at each of 0° C., −10° C., or −20° C. Long term storage, for example 64 days at −10° C. or −20° C., produced precipitates that could not be redissolved, even after warming to room temperature (15-25° C., or about 20° C.). Hence concentrates, or even diluted forms, of TOFA-derived corrosion inhibitors as conventionally used may be more unstable than desired at colder temperatures. Such precipitates or other phase separation exhibited by the concentrates is highly undesirable, since it render the concentrates difficult or impossible to pour or pump, either in cold temperatures or even at 20° C. or above if they concentrate has experienced irreversible cold-induced precipitation or other phase separation.

Without being bound by theory, it is conceivable that this instability may be the result, direct or indirect, of rosin content in the TOPA.

While the rosin acid content could be removed using a separation technique, a further drawback is much harder to overcome. It is becoming increasingly difficult to obtain tall oil or TOFA materials from commercial sources at prices that are economically practical. Accordingly, it has become increasingly desirable to find fatty amide materials that can be more easily sourced as an alternative to tall oil or TOFA materials.

Imidazolines may also be made by the reaction of vegetable oils with one or more amines, as shown, for example, in Schemes 2 and 3.

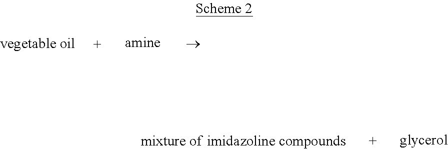

Alternatively, one or more vegetable oils may be hydrolyzed to produce hydrolyzed vegetable oil, which includes a mixture of fatty acids with byproduct glycerol. The hydrolyzed vegetable oil (fatty acids and glycerol) may then be reacted with an amine to produce fatty amides in combination with the glycerol. The reactions may be summarized as follows:

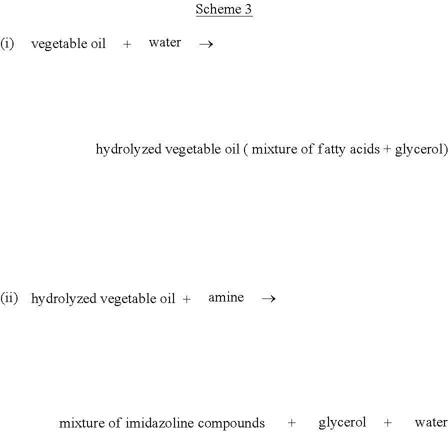

In Schemes 2 and 3 other components may be present. For example, the hydrolysis of a vegetable oil represented by Scheme 3, reaction (i) may be catalyzed by acid or base. It will be further understood that depending upon pH, the fatty acids may be present as salts thereof.

Imidazolines derived from vegetable oils show great potential as alternative dispersants to imidazolines derived from tall oil or TOFA. As one advantage, imidazolines derived from one or more vegetable oils can provide excellent dispersant and/or antifoulant properties, and as a natural matter contain de minimis if any rosin acid content. Unfortunately, a technical challenge against use of such materials relates to stability. Compositions that incorporate imidazolines derived from vegetable oils tend to have poor stability in terms of undergoing phase separation.

As can be seen from Schemes 2 and 3, production of imidazoline additives from vegetable oil provides a reaction product that includes glycerol. Imidazoline products derived from the reaction of a vegetable oil and amine may be prone to phase separation. Without wishing to be bound by theory, we believe that the presence of byproduct glycerol in the imidazoline products furnishes an additive composition that is more prone to phase separation, more specifically those derived from soybean oil and 2-aminoethylethanolamine (NH₂CH₂CH₂NHCH₂CH₂OH).

However, we have discovered that imidazoline additives derived from reacting amine compounds with glycerol-restricted soybean oil fatty acid are much more stable and resistant to phase separation. The preparation of a stable fatty imidazoline product may be represented by Scheme 4 (iii) and glycerol-restricted vegetable oil fatty acid may be produced by steps (i) and (ii). In step (ii), glycerol is removed from the hydrolyzed vegetable oil. In accordance with the present invention, reduced-glycerol imidazoline additives may be made in accordance with a reaction as illustrated, for example, in Scheme 4:

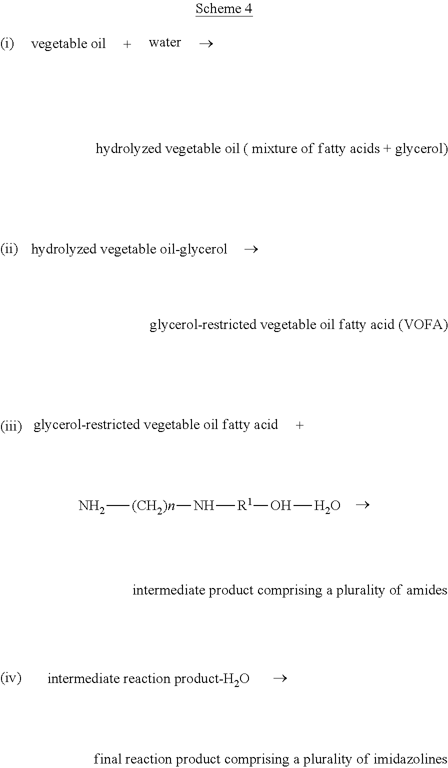

Significantly, the practice of the present invention provides a technical solution that makes the vegetable oil-derived imidazoline compounds much more stable. As a consequence, the imidazoline materials of the present invention may be used as a substitute for all or a portion of the tall oil and TOFA materials that have been used previously.

Glycerol-restricted soybean oil fatty acid (SOFA) is commercially available. Glycerol-restricted vegetable oil fatty acids, including those derived from soybean oil, may be produced by the following procedure: To one or more vegetable oils is added an alkali such as aqueous sodium hydroxide and/or aqueous potassium hydroxide to produce a mixture thereof. The mixture may be heated to a suitable temperature (such as in the range from 30° C. to 100° C., for example 30° C. to 100° C., or 40° C. to 90° C., or 50° C. to 100° C., or 60° C. to 100° C., or 50° C. to 90° C., or 50° C. to 80° C., or 50° C. to 70° C., or 55° C. to 65° C., or about 60° C.) for a suitable time (such as from one hour to 24 hours, for example 1 hour to 10 hours, or two hours to seven hours, or three hours to six hours, or three hours to five hours, or about four hours) effective for the saponification of the one or more vegetable oil to occur to the desired degree of completion. The saponification, the hydrolysis of the one or more vegetable oils, produces fatty acid salts and byproduct glycerol. To generate free fatty acids from the fatty acid salts, the pH of the mixture is reduced by adding a mineral acid such as sulfuric acid, hydrochloric acid, or a combination thereof. For example, the pH may be reduced to one to four, or one to three, or about two. An aqueous phase comprising water, a salt of the mineral acid, and glycerol separates from the organic phase into a distinct layer and the aqueous phase is separated from the organic phase. The organic phase may be dried. The organic phase may be further purified by separation into fractions, each fraction having a different range of acid numbers. For the present invention, the glycerol-restricted vegetable oil fatty acid may be derived from a fraction having an acid number of 170 to 230 mgKOH/g, or 180 to 220 mgKOH/g, or 190 to 210 mgKOH/g, or about 192 to 205 mgKOH/g. For example, the glycerol-restricted vegetable oil fatty acid may comprise, consist of, or consist essentially of glycerol-restricted soybean oil fatty acid having an acid number of about 192 to about 205 mgKOH/g.

The reaction between 2-aminoethylethanolamine and glycerol-restricted vegetable oil fatty acid, such as glycerol-restricted soybean oil fatty acid, may be represented as in Scheme 5, wherein R—COOH represents a mixture of fatty acids, each of which has a different R group;

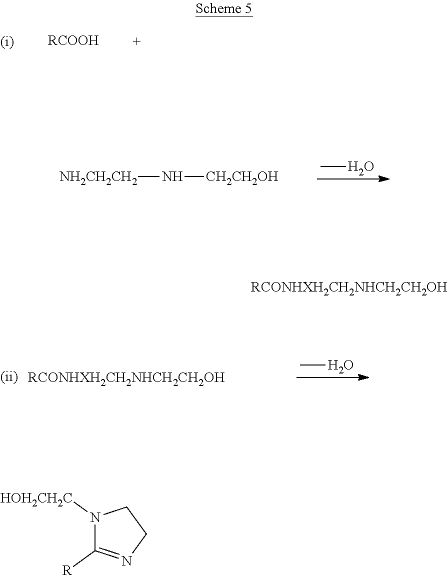

The composition of the mixture of fatty acids represented by RCOOH is characteristic of the type of vegetable oil fatty acid. For example, glycerol-restricted soybean oil fatty acid is different from glycerol-restricted corn oil fatty acid with respect to the types and/or relative proportions of individual fatty acids.

Methods of making additive compositions disclosed herein may provide imidazoline reaction products that comprise little or no glycerol, no rosin acids and/or their derivatives, and little or no reaction products of glycerol and resin acids, for example no more than 0.1 weight percent (percent by weight) thereof with respect to the total weight of imidazoline compounds in the reaction product. The methods use glycerol-restricted vegetable oil fatty acids, which are reacted with amine compounds in accordance with Scheme 5 to produce a combination of imidazoline compounds that is restricted in its glycerol content. The use of glycerol-restricted vegetable oil fatty acids avoids rosin acid content such as rosin acid or its derivatives. Using tall oil fatty acids, while it may restrict glycerol content, produces additive compositions that comprise rosin and/or its derivatives.

The use of glycerol-restricted vegetable oil fatty acids as opposed to tall oil or tall oil fatty acid avoids rosin acid content such as rosin acid or its derivatives. Rosin acid content is a natural component of the tall oils and tall oil fatty acids. Being acidic, rosin acid may have a tendency to be a corrodent in refinery equipment. Hence, avoiding rosin acid is highly desirable. Advantageously, soybean oil and many other vegetable oils have little if any rosin acid content. Consequently, the fatty amides and fatty esters derived from soybean oil and other vegetable oils avoid the corrosion risk associated with rosin acids. Unfortunately, though, additives derived from vegetable oil are prone to phase separation, thereby reducing the appeal of using such additives. However, the present invention teaches how the stability of these materials can be improved, making their use practical and desirable. The significant result is that the present invention not only avoids rosin acid corrodents but allows more stable vegetable oil derived materials to be used for corrosion protection.

### First Modes of the Invention

These modes illustrate glycerol-restricted fatty acid imidazolines that can be derived from one or more vegetable oils and then used as an additive in additive compositions for a variety of purposes such as dispersants in industrial systems such as industrial chemical facilities handling or processing unsaturated monomers. Accordingly, in first modes of the invention there is provided an additive composition comprising, consisting of, or consisting essentially of a plurality of imidazoline compounds. At least a portion of the plurality of the imidazoline compounds comprises, consists of, or consists essentially of a reaction product derived from reactants comprising, consisting of, or consisting essentially of one or more glycerol-restricted vegetable oil fatty acids and an amine.

The reaction product comprises, consists of, or consists essentially of imidazoline compounds. As set forth in Scheme 5, the reaction of amine and a glycerol-restricted vegetable oil fatty acid may produce reaction product (comprising, consisting of, or consisting essentially of imidazoline compounds) and byproduct water. Preferably, at least some of the byproduct

water is separated from the reaction product. Conveniently, at least a portion or even most or all of the byproduct water may be removed during the reaction. Removal of the water during the reaction assists in moving the imidazoline-forming reaction forward. Accordingly, the dried reaction product may comprise, consist of, or consist essentially of imidazoline compounds. In this context, “consist essentially” of allows for the presence of a small amount of water in the imidazoline compounds: even if dried or partially dried, the imidazoline compounds may include a small amount of residual water from the reaction and/or some water taken from the environment. The reaction product may comprise about 0 percent by weight to 1 percent by weight water, or about 0 percent by weight to 5 percent by weight water, or about 0 percent by weight to 2 percent by weight water, or about 0 percent by weight to 3 percent by weight water, or about 0 percent by weight to 4 percent by weight water, or about 0.5 percent by weight to 5 percent by weight water, or about 0.5 percent by weight to 4 percent by weight or about 0.5 to 3 percent by weight, or about 0.5 percent by weight to 2 percent by weight, or about 0.5 percent by weight to 1 percent by weight water, or about 0 percent by weight to 10 percent by weight, or about 0 percent by weight to about 20 percent by weight, or about 0.5 percent by weight to about 10 percent by weight, or about 0.5 percent by weight to about 20 percent by weight water with respect to the weight of the total imidazoline content in the reaction product.

Preferably, the reaction product comprises mostly or exclusively imidazoline compounds, and comprises no or a minimal amount of glycerol, no or a minimal amount of rosin acid, and no or a minimal amount of amide content. The reaction product may comprise 0 percent by weight to 1 percent by weight or 0 percent by weight to 2 percent by weight, or 0 percent by weight to 3 percent by weight, or 0 percent by weight to 4 percent by weight, or 0 percent by weight to 0.1 percent by weight, or 0 percent by weight to 0.01 percent by weight, or 0 percent by weight of a total of each of the following types of material with respect to the weight of the total imidazoline content in the reaction product: rosin acid, salt of rosin acid, amide of rosin acid, ester of rosin acid, fatty acid ester, and C1-C10 monohydric alcohol.

Because the cyclization reaction to for imidazoline compounds proceeds via amide intermediates, it is possible that the reaction product comprises some uncyclized fatty amide. The additive composition and/or the reaction product may comprise 0 percent by weight to 15 percent by weight of fatty acid amide, or 0.01 percent by weight to 12 percent by weight, or 0.1 percent by weight to 10 percent by weight, or 0 percent by weight to 10 percent by weight, or 0 percent by weight to 5 percent by weight, or 0 percent by weight to 2 percent by weight, or about 0 percent by weight, or 0.01 percent by weight to 0.1 percent by weight, or 0.01 percent by weight to 1 percent by total weight of fatty acid amide with respect to the weight of the total imidazoline content in the additive composition.

At least a portion of the plurality of imidazoline compounds comprises, consists of, or consists essentially of the reaction product. The reaction product may be used without further purification to provide an additive composition, purified further, or additional imidazoline compounds may be combined with the reaction product to form the plurality of the imidazoline compounds, with the proviso that the additional imidazoline compounds are free or relatively free of glycerol, rosin acid, or derivatives of them. The plurality of imidazoline compounds may comprise 80% to 100% by weight of the reaction product, or 85 percent by weight to 99 percent by weight, or 90 percent by weight to 99 percent by weight, or 94 percent by weight to 98% by weight, or 95 percent by weight to 97 percent by weight, or 95 percent by weight to 98 percent by weight, or 94 percent by weight to 96 percent by weight, or 96 percent by weight to 97 percent by weight, or 95 percent by weight to 96 percent by weight, or 94 percent by weight to 95 percent by weight, or about 100% by weight of the reaction product with respect to the weight of the plurality of imidazoline compounds.

The reactants may comprise, consist of, or consist essentially of one or more glycerol-restricted vegetable oil fatty acids; and one or more amine compounds. The one or more amine compounds comprises, consists of, or consists essentially of amine compounds satisfying the formula NH₂(CH₂)nNHR¹OH, wherein n is 2 or 3. In preferred embodiments, the one or more amine compounds may comprise, consist of, or consist essentially of 2-aminoethylethanolamine (AEEA) (NH₂CH₂CH₂—NH—CH₂CH₂OH).

The one or more glycerol-restricted vegetable oil fatty acids may be selected from one or more of glycerol-restricted avocado oil fatty acid, canola oil fatty acid, corn oil fatty acid, cottonseed oil fatty acid, grape seed oil fatty acid, hazelnut oil fatty acid, hemp seed oil fatty acid, linseed oil fatty acid, olive oil fatty acid, palm kernel oil fatty acid, peanut seed oil fatty acid, rape seed oil fatty acid, rice bran oil fatty acid, safflower oil fatty acid, sesame oil fatty acid, glycerol-restricted soybean oil fatty acid, sunflower seed oil fatty acid, and walnut oil fatty acid. In some preferred embodiments and aspects disclosed herein, the one or more glycerol-restricted vegetable oil fatty acids may comprise, consist of, or consist essentially of glycerol-restricted soybean oil fatty acid.

The one or more glycerol-restricted vegetable oil fatty acids may comprise 0 percent by weight to 0.01 percent by weight, or 0 percent by weight to 0.02 percent by weight, or 0 percent by weight to 0.03 percent by weight, or 0 percent by weight to 0.05 percent by weight, or 0.05 percent by weight to 0.1 percent by weight, or about 0 percent by weight of glycerol with respect to the total weight of the one or more glycerol-restricted vegetable oil fatty acids.

The one or more glycerol-restricted vegetable oil fatty acids may comprise, consist of, or consist essentially of glycerol-restricted soybean oil fatty acid. While the composition of soybean oil, as a natural product, may vary, the composition of soybean oil tends to fall into certain ranges. Accordingly, hydrolysis of soybean oil produces a plurality of fatty acids which also tends to fall within certain ranges of fatty acid content. The glycerol-restricted soybean oil fatty acid may comprise, consist of, or consist essentially of about 2 percent by weight to 15 percent by weight alpha-linolenic acid, about 46 percent by weight to about 56 percent by weight linoleic acid, about 17 percent by weight to about 28 percent by weight oleic acid, about 1 percent by weight to about 10 percent by weight stearic acid, and about 5 percent by weight to about 15 percent by weight palmitic acid and/or respective salts thereof with respect to the weight of the glycerol-restricted soybean oil fatty acid. For clarity, the weight of the glycerol-restricted soybean oil fatty acid includes the glycerol content, if any.

### Second Modes of the Invention

In second modes of the invention, there is provided a concentrate comprising, consisting of, or consisting essentially of (1) one or more organic solvents and (2) any of the additive compositions, reaction products, imidazoline compounds, and/or pluralities of imidazoline compounds as described hereinabove in the first modes of the invention. The additive compositions, reaction products, imidazoline compounds, and/or pluralities of imidazoline compounds are dissolved, dispersed, and/or otherwise in admixture with the one or more organic solvents, thereby providing a convenient means for addition to chemical mixtures, for example to facilitate dispersion of one or more components of the chemical mixture.

The one or more organic solvents may be selected from esters, alcohols, hydrocarbons, and combinations thereof. In preferred embodiments, the one or more organic solvents are liquid at 20° C. and one atmosphere pressure. The hydrocarbons may be selected from one or more of aromatic compounds, alkanes, heavy aromatic naphtha, toluene, xylene (o-xylene, m-xylene, p-xylene, or combination thereof) and alkenes. Examples of suitable hydrocarbons include but are not limited to benzene, toluene, C5 to C17 alkanes, alkenes, and naphtha. Examples of esters include but are not limited to phthalate esters such as dimethyl phthalate, diethyl phthalate, a dipropyl phthalate, a dibutyl phthalate, and combinations thereof.

The weight ratio of the additive composition, reaction product, concentrate, or pluralities of imidazoline compounds to the one or more organic solvents may be 3:7 to 2:3, or 2:3 to 1:1, or 1:1 to 3:2, or 3:2 to 7:3, or 7:3 to 4:1, or 4:1 to 9:1, or 5:1 to 3:1 respectively. The concentrate may comprise, consist of, or consist essentially of (1) the one or more organic solvents and (2) the additive composition, reaction product, concentrate, or pluralities of imidazoline compounds; or the concentrate may comprise, consist of, or consist essentially of the (1) the one or more organic solvents, (2) further components, and (3) the additive composition, reaction product, concentrate, or pluralities of imidazoline compounds. The additive composition may be any additive composition disclosed herein or a combination of any of the additive compositions disclosed herein.

The concentrate may comprise 30-40% solids by weight with respect to the weight of the concentrate, or 40-50% solids by weight, or 50-60% solids by weight, or 60-70% solids by weight, or 70-80% solids by weight, or 80-90% solids by weight, or 80-95% solids by weight, or 85-95% solids by weight, or 30-95% solids by weight, or 20-50% solids by weight, or 50-95% solids by weight with respect to the weight of the concentrate,

### Third Modes of the Invention

In third modes of the invention is provided an additive composition comprising, consisting of, or consisting essentially of one or more ammonium salts of any of the plurality of imidazoline compounds as described hereinabove in the first modes. The resultant ammonium salts may be incorporated into additive compositions, such as dispersant compositions, optionally in combination with water, optionally one or more additional optional ingredients, and combinations of these. The ammonium salts and aqueous solutions and/or dispersions thereof are particular useful for addition to aqueous chemical compositions, for example as dispersants or to impart other desirable properties to the aqueous chemical compositions.

The plurality of ammonium salts may be made by reacting the plurality of imidazoline compounds with one or more of an inorganic acid, an organic acid, an alkyl halide, and an aryl halide.

The inorganic acid may comprise, consist of, or consist essentially of hydrochloric acid. The organic acid may comprise, consist of, or consist essentially of acetic acid, acrylic acid, methacrylic acid, or a combination thereof. The alkyl halide may comprise, consist of, or consist essentially of an alkyl chloride. The aryl halide may comprise, consist of, or consist essentially of an aryl chloride.

In general, the reaction between acid and the plurality of imidazoline compounds may be summarized as in Scheme 6:

### Scheme 6

Plurality of imidazoline compounds+acid(aq)→plurality of ammonium salts(aq)

Similarly, one or more of the amine groups of the plurality of imidazoline compounds may be converted to alkyl or aryl ammonium groups by reaction with an alkyl halide or an aryl halide, preferably a C1 to C3 alkyl halide or an aryl halide.

In general, the reaction between a hydrocarbyl halide and the plurality of imidazoline compounds may be summarized as:

### Scheme 7

Plurality of imidazoline compounds+hydrocarbyl halide→plurality of N-hydrocarbyl imidazoline ammonium salts

While the additive compositions comprising, consisting of, or consisting essentially of imidazoline compounds are especially useful, for example, as additives to organic media, the ammonium salts disclosed herein are especially useful as additives for aqueous process streams and other aqueous media, for example as aqueous dispersants.

Any of the additives and concentrates of the first, second, and third modes may further comprise one or more of antipolymerants, antioxidants, and amine compounds.

### Fourth Modes of the Invention

In fourth modes of the invention there is provided a method of making the first additive as described hereinabove with respect to Schemes 4 (iii)-(iv) and 5. The method comprises: (1) combining one or more glycerol-restricted vegetable oil fatty acids with one or more amine compounds to form a reaction mixture, optionally but preferably at such a rate that the temperature of the reaction mixture does not exceed 100° C. The one or more glycerol-restricted vegetable oil fatty acids comprise, consist of, or consist essentially of fatty acids, and may be one or more hydrolyzed vegetable oils that are at least partially purified to remove glycerol.

The molar ratio of the one or more glycerol-restricted vegetable oil fatty acids to the one or more amine compounds in the reaction mixture may be 3:1 to 1:3, or 2:1 to 1:2, or 3:2 to 2:3, or 6:5 to 5:6, or about 1:1, or 1:1. While the one or more glycerol-restricted vegetable oil fatty acids comprises a mixture of fatty acid compounds, for calculating the aforementioned molar ratios, the acid number of the one or more glycerol-restricted vegetable oil fatty acids may be used to calculate a molecular weight for the one or more glycerol-restricted vegetable oil fatty acids.

The method optionally comprises: (2) a first heating step, the first heating step comprising, consisting of, or consisting essentially of heating the reaction mixture for a first time period at a first temperature, optionally under an inert gas such as nitrogen, wherein the first temperature is between 150° C. and 200° C., or 160° C. and 190° C., or 160° C. and 175° C., or 165° C. and 170° C., or about 180° C.; and wherein the first time period is between 30 minutes and 20 hours, or one hour and 15 hours, or one hour and ten hours, or five hours and 15 hours, or seven hours to nine hours, or five hours to ten hours, or about eight hours.

The method comprises a second heating step (3), wherein the second heating step comprises, consist of, or consists essentially of heating the reaction mixture for a second period of time at a second temperature, wherein the second period of time is 30 minutes to 20 hours, or 30 minutes to ten hours, or two hours to seven hours, or three hours to seven hours, or about five hours; and wherein the second temperature is 200° C. to 300° C., or 210° C. to 250° C., or 220° C. to 240° C., or 220° C. to 230° C., or about 225° C.

Preferably, the method further comprises (4) removing water from the reaction mixture during the first and/or second beating steps, for example by trapping the water during the heating in a Dean-Stark trap.

Any of the imidazoline additives of the first or second modes of the invention may be used in the synthesis or other processing of vinyl compounds such as (meth)acrylic acid.

In an exemplary embodiment, to a 250 mL four-necked round-bottom flask equipped with a temperature probe, nitrogen inlet, Dean-Stark apparatus, condenser, and magnetic stirrer bar is added glycerol-restricted soybean oil fatty acid (70.99 parts by weight). Next, 2-aminoethylethanolamine (29.00 parts by weight) is charged to the well-stirred reaction mixture. The temperature of the reaction mixture rises from 21° C. to about 57° C. The reaction is heated to about 180° C. and held for about eight hours. Next, the reaction is heated to 225° C. and held at 225° C. for five hours. About 4 mL of water is collected in the Dean-Stark trap. The reaction is cooled to below 100° C., and the reaction product transferred to a jar.

### Fifth Modes of the Invention

In fifth modes of the invention, there is provided a method of preventing fouling in a batch or process stream comprising a free radically polymerizable monomer, the method comprising, consisting of, or consisting essentially of adding to the batch or process stream an effective antifouling amount of any one or more of the additive compositions, reaction products, concentrates, or pluralities of imidazoline compounds that comprise one or more imidazoline compounds, as described in the first to fourth modes of the invention as disclosed herein.

The one or more of the additive compositions, reaction products, concentrates, or pluralities of imidazoline compounds may be added to the batch or process stream in the amount of 1-10,000 parts by weight of the one or more of the additive compositions, reaction products, concentrates, or pluralities of imidazoline compounds to one million parts by weight of the batch or process stream, or 10 ppm to 5000 ppm, or 10 ppm to 1000 ppm, or 10 ppm to 500 ppm, or 20 ppm to 500 ppm, or 30 ppm to 300 ppm by weight of the one or more of the additive compositions, reaction products, concentrates, or pluralities of imidazoline compounds with respect to the weight of the batch or process stream.

The composition of the batch or process stream may comprise one or more of: (a) one or more free radically polymerizable monomers, such as (meth)acrylic acid; (b) one or more polymerization inhibitors; and (c) one or more organic solvents; with the proviso that the composition comprises the one or more free radically polymerizable monomers. The one or more free-radically polymerizable monomers may comprise, consist of, or consist essentially of one or more of acrylic acid, methacrylic acid, one or more salts of acrylic acid, one or more salts of methacrylic acid, acrolein, one or more esters of acrylic acid, one or more esters of methacrylic acid, vinyl acetate, acrylonitrile, and methacrylonitrile. The one or more free radically polymerizable monomers may comprise, consist of, or consists essentially of one or more of (a) acrylic acid, (b) methacrylic acid, (c) one or more salts of acrylic acid, (d) one or more salts of methacrylic acid, and (e) vinyl acetate.

However, the additive compositions, reaction products, concentrates, or pluralities of imidazoline compounds of the first, second, and/or third modes of the invention as described herein may be added to other industrial material batches and/or process streams, for example to prevent fouling and/or act as a dispersant.

Any of the additive compositions, reaction products, concentrates, and pluralities of imidazoline compounds that comprise one or more imidazoline compounds and that are described herein may be used in compositions and methods of fouling in processes involving unsaturated monomers in the manner described, for example, in U.S. Pat. No. 7,005,0087 B2, which is incorporated herein by reference in its entirety and for all purposes. Further, any of the additive compositions, reaction products, concentrates, and pluralities of imidazoline compounds that comprise one or more imidazoline compounds and are described herein may be used in compositions and methods in downstream oil processes in a manner described, for example, in U.S. Pat. No. 3,766,053, which is incorporated herein by reference in its entirety and for all purposes.

### Sixth Modes of the Invention

Any of the additive compositions, reaction products, concentrates, or pluralities of imidazoline compounds that comprise one or more imidazoline compounds and are described in first to fifth modes of the invention herein may be added to chemicals and chemical compositions in order to impart desirable properties thereto. For example, the additive compositions, reaction products, concentrates, or pluralities of imidazoline compounds that comprise one or more imidazoline compounds may be added to a chemical composition comprising one or more unsaturated monomers in order to act as a dispersant for any undesirably formed polymer. Such treated compositions may show reduced fouling and settling when the imidazoline(s) in the additive compositions, reaction products, concentrates, or pluralities of imidazoline compounds act as a dispersant for any polymer and/or oligomer formed in the chemical composition.

Accordingly, in sixth modes of the invention is provided a treated composition comprising, consisting of, or consisting essentially of (a) any of the additive compositions, reaction products, concentrates, or pluralities of imidazoline compounds that comprise one or more imidazoline compounds and are described in first to fifth modes of the invention herein, and (b) one or more free-radically polymerizable monomers. The one or more free-radically polymerizable monomers may comprise, consist of, or consist essentially of one or more of acrylic acid, methacrylic acid, one or more salts of acrylic acid, one or more salts of methacrylic acid, acrolein, one or more esters of acrylic acid, one or more esters of methacrylic acid, vinyl acetate, acrylonitrile, and methacrylonitrile. The one or more free radically polymerizable monomers may comprise, consist of, or consists essentially of one or more of (a) acrylic acid, (b) methacrylic acid, (c) one or more salts of acrylic acid, (d) one or more salts of methacrylic acid, and (e) vinyl acetate.

The ratio of the weight of total imidazoline content to the weight of the treated composition may be 1-10,000 parts by weight of the total imidazoline content to one million parts by weight of the treated composition, or 10 ppm to 5000 ppm, or 10 ppm to 1000 ppm, or 10 ppm to 500 ppm, or 20 ppm to 500 ppm, or 30 ppm to 300 ppm by weight of total imidazoline additive composition with respect to the weight of the treated composition.

### EXAMPLES

The following examples are intended to illustrate different aspects and embodiments of the invention and are not to be considered limiting the scope of the invention. It will be recognized that various modifications and changes may be made without departing from the scope of the claims.

### Example 1: Synthesis of SOFA-2-aminoethylethanolamine (AEEA) Imidazoline Compounds from Glycerol-Restricted Soybean Oil Fatty Acid Obtained from Supplier A

To a 250 mL four-necked round-bottom flask equipped with a temperature probe, nitrogen inlet, Dean-Stark apparatus, condenser, and magnetic stirrer bar was added glycerol-restricted soybean oil fatty acid (70.99 g) obtained from a first supplier, Supplier A. Next, 2-aminoethylethanolamine (29.00 g, 0.278 moles) was charged to the well-stirred reaction mixture. The temperature of the reaction mixture was observed to rise from 21° C. to about 57° C. The reaction was heated to about 180° C. and held for about eight hours. Next, the reaction was heated to 225° C. and held at 225° C. for five hours. About 4 mL of water was collected in the Dean-Stark trap. The reaction was cooled to below 100° C., and transferred to a jar. The product remained a clear liquid and showed no signs of phase separation over time.

### Example 2: Synthesis of SOFA-2-aminoethylethanolamine (AEEA) Imidazoline Compounds from Glycerol-Restricted Soybean Oil Fatty Acid Obtained from Supplier B

To a 250 mL four-necked round-bottom flask equipped with a temperature probe, nitrogen inlet, Dean-Stark apparatus, condenser, and magnetic stirrer bar was added glycerol-restricted soybean oil fatty acid (71.31 g) obtained from a second supplier, Supplier B. Next, 2-aminoethylethanolamine (28.96 g, 0.275 moles) was charged to the well-stirred reaction mixture. The temperature of the reaction mixture was observed to rise from 20° C. to about 48° C. The reaction was heated to about 180° C. and held for about eight hours. Next, the reaction was heated to 225° C. and held at 225° C. for five hours. About 4.5 mL of water was collected in the Dean-Stark trap. The reaction was cooled to below 100° C. and transferred to a jar. The product remained a clear liquid and showed no signs of phase separation over time.

### Example 3: Imidazoline Additive from Soybean Oil and Diethylene Tetramine

To a one-liter four-necked round-bottom flask equipped with a temperature probe, nitrogen inlet, Dean-Stark apparatus, condenser, and magnetic stirrer bar was added 343.5 g of soybean oil with a few drops (0.01 g) of antifoaming silicone. The flask and its contents were heated to 80° C. under a nitrogen purge and diethylene tetramine (129.99 g, 1.26 moles) was added slowly in order that the contents of the flask did not exceed 100° C. and to avoid foaming. After the addition was completed, the contents of the flask were heated at 165° C. for an hour to form the amide, and the reaction mixture was monitored for formation of the amide by removing small samples and checking them by infrared spectroscopy for the disappearance of the peak associated with ester carbonyl at 1742 cm⁻¹ and the appearance of the peak associated with amide carbonyl at 1645 cm⁻¹.

The contents of the reaction flask were then heated to 245° C. for three hours to cyclize amide to imidazoline. The reaction was monitored for formation of the imidazoline by removing small samples and checking them by infrared spectroscopy for the disappearance of the amide carbonyl peak at 1645 cm⁻¹ and the appearance of the imine peak at 1602 cm⁻¹.

The contents of the reaction vessel were cooled and transferred to a jar. Immediately after transfer, the contents of the jar were a clear liquid, but after three days the contents of the jar were turbid and had phase separated into layers.

### Example 4: Imidazoline Additive Concentrate from Glycerol-Restricted Soybean Oil Fatty Acid

To a 250 mL four-necked round-bottom flask equipped with a temperature probe, nitrogen inlet, Dean-Stark apparatus, condenser, and magnetic stirrer bar was added glycerol-restricted soybean oil fatty acid (70.99 g). Next, 2-aminoethylethanolamine (29.00 g, 0.278 moles) was charged to the well-stirred reaction mixture. The temperature of the contents of the flask rose from room temperature to a temperature between about 50 and about 60° C. The viscous reaction mass was heated at about 180° C. for about eight hours. The reaction mixture was heated then heated to about 225° C. and held at that temperature for about five hours. About four to five mL of water was collected in the Dean-Stark trap. The contents of the flask were cooled to less than 100° C., and transferred to a jar.

A sample of the reaction product, SOFA-imidazoline, was stored at −10° C. and did not exhibit haze or a precipitate after 11 days.

A sample of the SOFA-imidazoline product (8 g) was blended with dimethyl phthalate (2 g) to form a concentrate. The concentrate was stored at −10° C., and showed no haze or precipitation after a week, after 44 days, or even after 95 days.

### Example 5: Imidazoline Additive Concentrate from Tall Oil Fatty Acid

The procedure of Example 4 was repeated, but instead of glycerol-restricted soybean oil fatty acid, tall oil fatty acid was used.

A sample of the reaction product, TOFA imidazoline, was stored at −10° C. for 11 days and exhibited a precipitate.

A sample of the TOFA-imidazoline product (8 g) was blended with dimethyl phthalate (2 g) to form a concentrate. The concentrate was stored at −10° C., and exhibited precipitation after only a few days. After storage at −10° C. for 64 days, the precipitate could not be easily redissolved.

Comparison with Example 4 shows that the SOFA-imidazoline additive was more stable when stored at low temperatures than the corresponding TOFA-imidazoline additive, both in 100% form and when made up as a concentrate in organic solvent (dimethyl phthalate).

### Example 6: Dispersant Performance of Imidazoline Concentrates with 2-Ethylhexyl Acrylate Foulant

Three samples were prepared by mixing components in a dispersant tube, Samples O, S, and T.

Two concentrates of imidazoline additives in dimethyl phthalate were made, concentrate S and concentrate T. Each concentrate was made by combining 8 parts by weight of the imidazoline additive with 2 parts by weight of dimethyl phthalate, Concentrate S comprised imidazoline compounds made from SOFA and AEEA, whereas concentrate T comprised imidazoline compounds made from TOFA and AEEA. Concentrate S was made as described in Example 4 and Concentrate T was made as in Example 5.

Sample O was prepared by mixing 2-ethylhexyl acrylate foulant (1 mL) with methyl methacrylate (10 mL) in a dispersant tube (similar to a test tube, but with a tapered lower end). Sample S was prepared by mixing by mixing 2-ethylhexyl acrylate foulant (1 mL), methyl methacrylate (10 mL), and 100 μL of Concentrate S in a dispersant tube. Sample T was made by mixing 2-ethylhexyl acrylate foulant (1 mL), methyl methacrylate (10 mL), and 100 μL of Concentrate T in a dispersant tube. All three Samples were made using the same source mixture of the 2-ethylhexyl acrylate and methyl methacrylate. The dispersant tubes were all mounted vertically in rack. In Sample O (without dispersant), the foulant was seen to have begun settling, even after only five minutes, whereas in Samples S and T the foulant remained dispersed and did not appear to settle. The tubes were observed after 40 minutes, 60 minutes, and 24 hours. After all of these times, the foulant appeared to remain dispersed throughout the sample in Samples S and T, whereas the foulant gradually settled out of Sample O leaving a clearer supernatant liquid above.

1. An additive composition comprising:
a plurality of imidazoline compounds and/or ammonium salts thereof,
wherein at least a portion of the plurality of imidazoline compounds comprises a reaction product derived from reactants comprising an amine and a glycerol-restricted soybean oil fatty acid,
wherein the glycerol-restricted soybean oil fatty acid comprises a mixture of fatty acids and less than 0.1 percent by weight glycerol with respect to the weight of the glycerol-restricted soybean oil fatty acid, and
wherein the amine has the formula NH₂—(CH₂)ₙ—NH—R¹—OH, wherein R¹ is a divalent moiety comprising one or more C atoms and optionally one or more heteroatoms, and wherein n is 2 or 3.

2. The additive composition of claim 1, wherein the additive composition comprises 0 percent by weight to 3 percent by weight of glycerol with respect to the weight of the additive composition.

3. The additive composition of claim 1, wherein R¹ has the formula (CH₂CH₂NH)ₓCH₂CH₂, wherein x is 1-100.

4. The additive composition of claim 1, wherein R¹ has the formula CH₂CH₂.

5. The additive composition of claim 1, wherein the additive composition comprises the plurality of the imidazoline compounds.

6. The additive composition of claim 1, wherein the glycerol-restricted soybean oil fatty acid comprises about 2 percent by weight to about 15 percent by weight alpha-linolenic acid, about 46 percent by weight to about 56 percent by weight linoleic acid, about 17 percent by weight to about 28 percent by weight oleic acid, about 1 percent by weight to about 10 percent by weight stearic acid, and about 5 percent by weight to about 15 percent by weight palmitic acid with respect to the weight of the glycerol-restricted soybean oil fatty acid.

7. The additive composition of claim 1, wherein the amine has the formula NH₂CH₂CH₂—NH—CH₂CH₂OH.

8. The additive composition of claim 1, wherein the additive composition comprises:
0 percent by weight to 10 percent by weight total amide content, 0 percent by weight to 5% rosin acid, 0 percent by weight to 5 percent by weight of a salt of rosin acid, 0 percent by weight to 5 percent by weight amide of rosin acid, 0 percent by weight to 5 percent by weight of ester of rosin acid, 0 percent by weight to 5 percent by weight total C1-C10 monohydric alcohol, and 0% to 5 percent by weight total fatty acid esters with respect to the weight of the plurality of imidazoline compounds, the plurality of ammonium salts of the imidazoline compounds, or the combination thereof.

9. The additive composition of claim 1, wherein the additive composition comprises 0.01 percent by weight to 10 percent by weight of total amide with respect to the weight of the additive composition.

10. The additive composition of claim 1, wherein the additive composition comprises 0 percent by weight of total amide with respect to the weight of the additive composition.

11. The additive composition of claim 1, further comprising:
about 0.1 parts by weight to 75 parts by weight of at least one organic solvent per 99.9 parts by weight to 25 parts by weight of the plurality of imidazoline compounds, the plurality of ammonium salts of the imidazoline compounds, or the combination thereof.

12. The additive composition of claim 11, the concentrate comprising about 10 parts by weight to about 30 parts by weight of the at least one organic solvent per 70 to 90 parts by weight of the plurality of imidazoline compounds, the plurality of ammonium salts of the imidazoline compounds, or the combination thereof.

13. The additive composition of claim 11, wherein the at least one organic solvent comprises one or more phthalates.

14. The additive composition of claim 11, wherein the at least one organic solvent comprises dibutyl phthalate, dimethyl phthalate, or a combination thereof.

15. The additive composition of claim 11, wherein the at least one organic solvent consists of dibutyl phthalate, dimethyl phthalate, or a combination thereof, and the composition does not comprise any other organic solvent.

16. The additive composition of claim 11, wherein the concentrate comprises 0 percent by weight to 2 percent by weight glycerol, 0 percent by weight to 2% rosin acid, 0 percent by weight to 2 percent by weight of a salt of rosin acid, 0 percent by weight to 2 percent by weight of an amide of rosin acid, 0 percent by weight to an ester of rosin acid, 0 percent by weight to 2 percent by weight total C1-C10 monohydric alcohol, and 0% to 2 percent by weight total fatty acid esters with respect to the weight of the concentrate.

17. A treated composition comprising:
(i) an additive composition comprising a plurality of imidazoline compounds and/or ammonium salts thereof, wherein at least a portion of the plurality of imidazoline compounds comprises a reaction product derived from reactants comprising an amine and glycerol-restricted soybean oil fatty acid,
wherein the glycerol-restricted soybean oil fatty acid comprises a mixture of fatty acids and 0 weight percent to 0.1 percent by weight glycerol with respect to the weight of the glycerol-restricted soybean oil fatty acid, and
wherein the amine satisfies the formula satisfies the formula NH₂—(CH₂)ₙ—NH—R¹—OH, wherein R¹ is a divalent moiety comprising one or more C atoms and optionally one or more heteroatoms, and wherein n is 2 or 3;
and
(ii) a free-radically polymerizable monomer.

18-22. (canceled)

23. A method of making an additive composition, the method comprising:
(a) combining an amine and glycerol-restricted soybean oil fatty acid to provide a reaction mixture; and
(b) heating the reaction mixture to form water and the additive composition, wherein the additive composition comprises a plurality of imidazoline compounds,
wherein at least a portion of the imidazoline compounds comprises a reaction product derived from reactants comprising the amine and the glycerol-restricted soybean oil fatty acid,
wherein the glycerol-restricted soybean oil fatty acid in step (a) comprises a mixture of fatty acids and comprises less than 0.1 percent by weight glycerol with respect to the weight of the glycerol-restricted soybean oil fatty acid in step (a), and
wherein the amine in step (a) satisfies the formula NH₂—(CH₂)ₙ—NH—R¹—Y, wherein R¹ is a divalent moiety comprising one or more C atoms and optionally one or more heteroatoms, wherein n is 2 or 3, and wherein Y is —NH₂ or —OH.

24. The method of claim 23, the method further comprising reacting the plurality of the imidazoline compounds with at least one acid to form a plurality of ammonium salts of imidazoline compounds.

25. The method of claim 24, wherein the at least one acid comprises one or more of hydrochloric acid, sulfuric acid, acetic acid, carbonic acid, acrylic acid, and methacrylic acid.

26. The method of claim 23, the method further comprising reacting the plurality of the imidazoline compounds with at least one alkyl halide, at least one aryl halide, or a combination thereof to form a plurality of ammonium salts of imidazoline compounds.

27. The method of claim 23, wherein the additive composition comprises 0 percent by weight to 3 percent by weight glycerol with respect to the weight of the additive composition.

28. The method of claim 23, wherein the amine has the formula NH₂CH₂CH₂—NH—CH₂CH₂OH.

29. The method of claim 23, wherein the glycerol-restricted soybean oil fatty acid comprises about 2 percent by weight to about 15 percent by weight alpha-linolenic acid, about 46 percent by weight to about 56 percent by weight linoleic acid, about 17 percent by weight to about 28 percent by weight oleic acid, about 1 percent by weight to about 10 percent by weight stearic acid, and about 5 percent by weight to about 15 percent by weight palmitic acid with respect to the weight of the glycerol-restricted soybean oil fatty acid.

30. The method of claim 23, wherein the reaction mixture comprises 98%-100% by weight of a combination of 2-aminoethylethanolamine and glycerol-restricted soybean fatty acid with respect to the weight of the reaction mixture.

31. The method of claim 23, wherein the additive composition comprises 0 percent by weight to 10 percent by weight total amide content, 0 percent by weight to 5% rosin acid, 0 percent by weight to 5 percent by weight of a salt of rosin acid, 0 percent by weight to 5 percent by weight of an ester of rosin acid, 0 percent by weight to 5 percent by weight total C1-C10 monohydric alcohol, and 0 percent by weight to 5 percent by weight total fatty acid esters with respect to the weight of the plurality of imidazoline compounds.

32. (canceled)
